(19) United States
(12) Patent Application Publication
(10) Pub. No.: US 2024/0122584 A1
(43) Pub. Date: Apr. 18, 2024

(54) ELECTRIC FIELD ASSISTED BIOPSY (EFAB)

(71) Applicant: University of Massachusetts, Boston, MA (US)

(72) Inventors: Govindarajan Srimathveeravalli, Amherst, MA (US); Mary Chase Sheehan, Northampton, MA (US); Yasushi Kimura, Osaka-fu (JP); Devan Kumar, Gardner, MA (US)

(21) Appl. No.: 18/484,712

(22) Filed: Oct. 11, 2023

Related U.S. Application Data
(60) Provisional application No. 63/379,084, filed on Oct. 11, 2022.

(51) Int. Cl. A61B 10/02

(52) U.S. Cl. CPC A61B 10/0266; A61B 10/0283; A61B 2010/0208

(57) ABSTRACT

A device may (i) at least one electrode associated with an electric generator to generate a pulsed electric field (PEF) in solid tissue, wherein said electrode comprises more than one hole/opening at the end in contact with said solid tissue and an insulating sleeve that can be adjusted to alter the side hole profile. A device may (ii) a cellular-component extraction element by suction through at least one electrode, wherein upon introducing said at least one electrode into said solid tissue, and generating the PEF, the PEF induces a biophysical response from cells in solid tissue or other condition (such as be blood, in vitro etc.) resulting in at least one cellular component to exit to extracellular matrix which is then extracted by said extraction element.

### CROSS-REFERENCE TO RELATED APPLICATIONS

This application claims the benefit of priority to U.S. Provisional Patent Application Ser. No. 63/379,084 entitled “ELECTRIC FIELD ASSISTED BIOPSY (EFAB),” filed Oct. 11, 2022, the disclosure of which is incorporated herein in its entirety by reference.

### GOVERNMENT GRANT SUPPORT

This invention was made with government support under W81XWH-19-PRCRP-IASF and W81XWH2010676 awarded by the Department of Defense and R01DK129990 and R01CA236615 awarded by the National Institutes of Health. The government has certain rights in the invention.

### BACKGROUND OF THE INVENTION

Currently available techniques for biopsy, such as high frequency focused ultrasound require sizeable equipment investment, planning and operation, and a needle must still be used acquire samples. Other techniques involve imaging, and yet, do not yield a sample that can be fed into existing clinical workflow. Vacuum and ultrasound assisted biopsies are unable to predict volume being surveyed or provide high specificity/sensitivity.

### BRIEF DESCRIPTION OF THE DRAWINGS

FIG. 1A-1B. Intra-tumoral heterogeneity shown with different colors. 1A) Sample from conventional biopsy (dashed line). 1B) EFAB allows volumetric profiling of intracellular contents released by cells throughout the tumor using EP (inset).

FIG. 2A-2H. 2A) In vitro setup for demonstrating EFAB in 3D tumor model. 2B-2D) Volume of tumor (purple) undergoing EP (red) at increasing energy dose. 2E) FEM model of in vitro setup, and 2F-2H).

FIG. 3. A perspective view of a device to perform EFAB.

FIGS. 4A-4E. Show 4A. comparison of conventional (top) and proposed EFAB waveform. 4B. Protein and 4C. RNA release with increasing pulse width. Cells loaded with dye in 4D. Sham and 4E. EFAB with proposed waveform.

FIGS. 5A-5D. Show 5A. Cells with 3D tumor model releasing material during EFAB at different voltages, mapped with PI dye. 5B. Correlative map of the electric field gradient and critical threshold marking the boundary. 5C. Dose escalation study with GFP+ cells and a central portion lacking dye. 5D. Quantity of dye in EFAB sample increases in proportion to the applied voltage.

FIGS. 6A-6C. Show 6A. An axisymmetric model of a spherical tumor with mesh. 6B. Electric field distribution at increasing voltage marks EFAB threshold. 6C. Corresponding flow pattern during suction with region of sampling.

FIG. 7. Shows simulation with heterogenous conductivity and anatomic features (left). Electric inhomogeneities when using low (right, top) and high frequency (right, bottom) pulses.

FIGS. 8A-8E. Show 8A. H&E tumor section. 8B. homogenous σ map and 8C. electric field 8D. heterogenous σ map, and corresponding 8E. electric field distribution.

FIGS. 9A-9B. Show CAD models of the medical device consumable for electric pulse delivery and sample acquisition.

FIGS. 10A-10E. Show a mechanical testing setup of the medical device.

FIGS. 11A-11C. Show 11A. PDMS device containing three 3D tumor mimic formed (10×10 mm) by embedding cancer cells in a collagen matrix. Arrows indicate location of the needles placed in the test gel for electric pulse delivery. 11B. Sham treated gel has diffuse, minimal fluorescence, that is 11C. increased in the test condition in the region between the electrodes where the electric pulses were delivered.

FIGS. 12A-12C. Show 12A. 3D model of the fixture for positioning patient prostate. 12B. Fixture with prostate. Needles are inserted via the template grid into two locations within the prostate. Electric pulses were delivered between the two electrodes and sample drawn through the same. 12C. Top view of the setup.

### SUMMARY

In some aspects, the techniques described herein relate to a device including: (i) at least one electrode associated with an electric generator to generate a pulsed electric field (PEF) in solid tissue, wherein said electrode includes more than one hole/opening at the end in contact with said solid tissue and an insulating sleeve that can be adjusted to alter the side hole (if present) profile; and (ii) a cellular-component extraction element by suction through at least one electrode, wherein upon introducing said at least one electrode into said solid tissue, and generating the PEF, the PEF induces a biophysical response from cells in solid tissue or other condition (such as be blood, in vitro etc.) resulting in at least one cellular component to exit to extracellular and/or interstitial space which is then extracted by said extraction element.

In some aspects, the techniques described herein relate to a method to extract biological material from a tissue of a subject, said method including: i) placing at least one electrode within said tissue, wherein said electrode includes one or more hole(s)/opening(s) at the end in contact with said tissue and an insulating sleeve that can be adjusted to alter the side hole profile; ii) applying pulsed electric field (PEF) via said at least one electrode of (i) to thereby induce permeabilization and/or other biophysical responses (such as cell stimulation) of cells of said tissue and release of at least one cellular component therefrom to an interstitial space; and iii) extracting said at least one cellular component from said interstitial space and optionally providing the extracted at least one cellular component to standard clinical diagnostic or biomedical research workflows, wherein one or more computational models are used to guide parameter selection to guide treatment and extraction parameters.

In some aspects, the techniques described herein relate to a method to determine if a tissue in a subject includes a tumor, said method including: i) placing at least one electrode within said tissue, wherein said electrode includes more than one hole/opening at the end in contact with said tissue and an insulating sleeve that can be adjusted to alter the side hole profile; ii) applying pulsed electric field (PEF) via said at least one electrode of (i) to thereby induce permeabilization and/or other biophysical responses (such as cell stimulation) of cells of said tissue and release of at least one cellular component therefrom to an interstitial space; iii) extracting said at least one cellular component from said interstitial space to feed into standard clinical diagnostic or biomedical research workflows; and iv) using the standard clinical diagnostic or biomedical research workflows to identify the at least one cellular component extracted so as to determine the presence of the tumor within said tissue, wherein one or more computational models are used to guide parameter selection to guide treatment and extraction parameters.

The emerging need for comprehensive tumor characterization: Tumors exhibit remarkable spatial heterogeneity in clonal cancer cells, protein expression and in the presence and functional status of immune cells. Such intratumoral heterogeneity (ITH) is linked to cancer progression and resistance to targeted drug therapy. Intratumoral levels of therapy related protein biomarkers (e.g., HER2, estrogen receptor, PD-L1) has been found to be inextricably linked to variations in cellular and genomic factors within the tumor microenvironment (TME). These clinical experiences suggest that solitary cancer biomarkers are no longer sufficient to guide clinical decision making. It is evident that in this era of precision oncology and personalized medicine, comprehensive extraction of multi-omic information from the entire tumor is an urgent clinical need to aid accurate diagnosis and guide optimal treatment strategies.

Surgically resected tumors are the definitive biological source for profiling the TME, enabling discoveries in ITH and spatial variations of cancer biomarkers, and understanding their impact on disease progression and treatment outcomes. However, surgical samples are largely unavailable from most cancer patients. Surgical resection is uncommon at preliminary disease diagnosis, and in the context of metastatic, disseminated disease. Moreover, patients undergoing surgical resection of their tumors typically have a favorable, localized disease profile; thereby limiting the diversity of biological samples available to inform clinical cancer research. Complete access to the tumor genome, transcriptome and proteome from diverse patient tumors and cancer stages would catapult our ability to study and combat cancer effectively. The fundamental bottleneck limiting further progress on this topic is our inability to non-surgically extract comprehensive tumor samples to fully utilize the state-of-the-art technologies currently available for molecular cancer diagnosis.

The inherent limitations of clinical biopsy for tumor sampling: The primary source of tumor specimens for cancer diagnosis and research are by biopsy. There are two forms of this procedure; core biopsy wherein a thin section of the tumor is extracted (<1 mm wide, and 5-20 mm long) and fine needle aspiration (FNA) where a loose collection of cells and fluids are retrieved. As needle biopsies are central to cancer care, professional medical societies and institutions have incorporated quality assurance practices to improve specimen quality. Despite optimization, biopsies are highly sensitive to cancer type, extraction order and physician technique. In a trial on biopsy of hepatocellular carcinoma, Weinfurtner et al. reported that despite retrieving several cores, fewer than 41% of samples were deemed adequate for proteomic or genomic analysis. Bhamidipati et al. reported a study with 1149 research biopsies covering 20 cancer types. They found that over 55% of the samples contained <10% of malignant content, thereby deemed inadequate for molecular diagnosis. The chance of obtaining an inadequate sample increased with the order in which the core was obtained, with sensitivity to patient related factors (age, disease status and biopsy location). Similar findings have been reported by other studies examining biopsy of lung, breast, and prostate cancer and in immunotherapy trials. Clinical biopsy techniques sample a small fraction of the total tumor volume, where the location of tissue acquisition is driven by non-oncologic factors such as the feasibility of percutaneous approach. Prior studies have defined biopsy adequacy for molecular diagnosis primarily based on the presence of sufficient malignancy related material for genomic or proteomic analysis, whereas the ability to accurately represent or capture the global TME has not been examined. The current paradigm of cancer diagnosis is predicated on what's seen on conventional biopsies, whereas clinical trials results are suggesting that treatment failure and disease progression may be driven by the critical information of the global TME missing in these samples.

The concept of electric field assisted biopsy: Microsecond long, high voltage pulsed electric fields (PEF) are routinely used with patients for tumor ablation, gene and drug delivery. Cells exposed to PEF experience rapid, transient alteration of their transmembrane potential leading to pore formation in the plasma membrane. This process is termed electroporation, where temporary membrane permeabilization (reversible electroporation, RE) enables drug and gene delivery, whereas permanent permeabilization leads to cell death (irreversible electroporation, IRE). Electroporation has an excellent clinical safety profile, where IRE is routinely used by physicians for the treatment of tumors adjacent to sensitive anatomical structures such as tubular organs (e.g., bile duct, bladder etc.) and nerves. This favorable safety profile is enabled by the working mechanism of electroporation that injects just a few watts of energy into the tissue, thereby avoiding damaging temperature changes in the treated area.

Prior work on using electroporation for intracellular material extraction: Permeabilization of the cell membrane during electroporation simultaneously allows the outward diffusion of intracellular contents such as DNA, RNA, metabolites, and proteins within seconds following PEF application. This well-known phenomenon has been previously tested for DNA extraction by lysis of cancer cells within microfluidic devices and is commonly used in the food industry for the extraction of nutrients from plant and fruit products. However, there have been no prior efforts to adapt this technique for the in vivo setting. Golberg et al have recently reported the use of conventional PEF parameters used for RE to extract intracellular materials from tumors. While this reinforces the feasibility of using PEF to enhance biopsy, our work provides foundational material for specific waveforms and companion computational models to establish the new field of EFAB.

The PEF parameters and waveforms for RE and IRE were established following decades of computational optimization and experimental validation. A rich complement of computational models describing electrical and heat transfer physics in tissue treated with PEF were essential for development of these applications. Computational models developed for RE or IRE applications are inherently incapable of guiding the design of pulse parameters for intracellular material release and lack physics equations necessary for mapping the effect of negative pressure gradients in the sampling process. The computational models we have described are therefore essential to advance the topic of EFAB, by establishing specific pulse sequences for optimal intracellular material extraction, defining synergistic parameters for the electric field and suction gradients that are necessary for the sampling process, and identifying specific PEF frequency to successfully perform EFAB under conditions of heterogenous intratumoral physical properties.

Prior work on imaging and mechanical techniques to improve biopsy sample acquisition: Imaging driven approaches and devices have been developed to improve our ability to characterize the global TME. Imaging can inherently capture ITH based on contrast, density gradients, diffusion patterns and vascularization. Over the past two decades radiomics and image-feature analysis has attempted to correlate ITH with underlying genomic alterations in tumors. This approach has been successfully evaluated in different cancer types, where outcomes have good agreement with pathology. The emergence of machine learning techniques has stimulated further investigations on this topic. Despite these positive outcomes, there exist certain insurmountable limitations. Imaging and radiomic techniques are highly sensitive to image acquisition conditions, presenting challenges in producing consistent and reproducible results. The number of molecular targets that can be evaluated through imaging techniques remain limited. Finally, tissue specimens are still required pathology and molecular diagnosis. Physical modifications to biopsy devices have been evaluated to improve sampling. These include the use of vacuum or vibrations to improve tissue acquisition. These improvements, however, do not address the fundamental inability to improve the tumor volume being profiled. Ultrasound based techniques have provided a unique opportunity to improve volumetric profiling of tumors. Chen et al. have shown the feasibility of Sonobiopsy to stimulate release of materials from brain tumors to generate liquid biopsies that can be extracted systemically. The broader applicability of US technologies is dampened by the considerable resource requirements for the technology, the potential for sample quality degradation and dilution during systemic collection of the liquid biopsy, as well as the difficulties in integrating the technique into clinical biopsy workflow.

The standard of care in management of cancer patients is to perform a biopsy, for example on a benign or malignant solid tumor, cyst or other mass, is with fine needle aspiration, where the volume from which sampling occurs is unpredictable. Further, contamination by blood pool (requiring additional steps prior to processing, such as for a molecular diagnosis) and inability to survey large volumes of tissue (greater 2-3 mm) are limitations to the art.

One embodiment provides a device comprising: (i) at least one electrode associated with an electric generator to generate a pulsed electric field (PEF) in solid tissue, wherein said electrode comprises more than one hole/opening at the end in contact with said solid tissue and an insulating sleeve that can be adjusted to alter the side hole profile; and (ii) a cellular-component extraction element by suction through at least one electrode, wherein upon introducing said at least one electrode into said solid tissue, and generating the PEF, the PEF induces a biophysical response from cells in solid tissue or other condition (such as be blood, in vitro etc.) resulting in at least one cellular component to exit to extracellular matrix which is then extracted by said extraction element.

Another embodiment provides a method to extract biological material from a tissue of a subject, said method comprising: i) placing at least one electrode within said tissue, wherein said electrode comprises more than one hole/opening at the end in contact with said tissue and an insulating sleeve that can be adjusted to alter the side hole profile; ii) applying pulsed electric field (PEF) via said at least one electrode of (i) to thereby induce permeabilization and/or other biophysical responses (such as cell stimulation) of cells of said tissue and release of at least one cellular component therefrom to an interstitial space; and iii) extracting said at least one cellular component from said interstitial space and optionally providing the extracted at least one cellular component to standard clinical diagnostic or biomedical research workflows, wherein one or more computational models are used to guide parameter selection to guide treatment and extraction parameters.

One embodiment provides a method to determine if a tissue in a subject comprises a tumor, said method comprising: i) placing at least one electrode within said tissue, wherein said electrode comprises more than one hole/opening at the end in contact with said tissue and an insulating sleeve that can be adjusted to alter the side hole profile; ii) applying pulsed electric field (PEF) via said at least one electrode of (i) to thereby induce permeabilization and/or other biophysical responses (such as cell stimulation) of cells of said tissue and release of at least one cellular component therefrom to an interstitial space; iii) extracting said at least one cellular component from said interstitial space to feed into standard clinical diagnostic or biomedical research workflows; and iv) using the standard clinical diagnostic or biomedical research workflows to identify the at least one cellular component extracted so as to determine the presence of the tumor within said tissue, wherein one or more computational models are used to guide parameter selection to guide treatment and extraction parameters.

Wherein the one or more computational models include those discussed herein, as well as computational models of physical phenomenon of PEF mediated electroporation, localized heating, change in tissue stiffness, cell death that will guide selection of electric pulse parameters (voltage, pulse width, number of pulses and frequency) to be applied at the electrodes to elicit intracellular material from desired tissue volumes or identify the volume of effect based on the parameter set and to determine the relative concentration of elicited material within the volume, and the gradient of distribution. These parameters linked to the computation model combining flow through porous media under suction, tissue deformation and stiffness calculations will identify suction parameters (strength, duration, duty cycle) required to extract material from defined volumes and map the electric field and suction models to determine the makeup of the lysate based on the combination of treatment and suction parameter set.

### DETAILED DESCRIPTION

Provided herein are devices, simulation models and algorithms for predictable, volumetric extraction of intracellular materials (DNA, RNA, protein, metabolites) from bulk tissue, including generation of on-demand, volumetric liquid biopsy from solid tumors. The invention allows one to survey patient tumors to improve cancer diagnosis, support immunotherapy prognosis, assess premalignant conditions and provides access to tissue samples in benign conditions without injury to organ.

Patients with primary or metastatic Renal Cell Carcinoma (mRCC) or other solid tumors, such as primary or metastatic cancers including lung, liver, pancreas colorectal, and kidney cancer, exhibit marked variations in disease aggressiveness and response to therapy, where a subset of patient succumb to cancer within months despite best-in-class treatments, yet others can have an indolent disease course that remains stable for years without intervention. It is therefore crucial to have the ability to comprehensively profile and characterize solid tumors such that clinical management plans can be tailored to match patient specific disease characteristics, assigning treatments to only those patients likely to respond or benefit from it.

While some patients show response to immunotherapy, others do not. For example, While PD-1/PD-L1 axis checkpoint inhibitor immunotherapy (CII) has been shown to substantially prolong patient survival, less than half of mRCC patients receiving CII demonstrate response despite being selected on basis of positive PD-L1 expression in their tumors. While the potential benefit of CII for patients with mRCC is clear, the clinical challenge is the lack of a biomarker or diagnostic technique that can improve patient stratification for assignment to undergo treatment. This is the case for all solid tumors and markers.

Provided herein is the technique of pulsed electric fields (PEF) and the feasibility of using it to extract intracellular material such as RNA and proteins for evaluation with standard clinical techniques. The application of PEF is scalable from single cell suspensions, adherent cells in cultures, experimental organoids, small and large animal models as well as patients. Devices are available or can be readily made to perform pulse delivery in vitro and in vivo, and simulation models can be used to choose pulse parameters to evoke specific cellular response. Further refining the PEF techniques will increase yield of intracellular material without adversely affecting cell survival or proliferation.

One embodiment provides devices and methods for extracting cellular components, e.g., proteins, RNA, DNA, and/or metabolites, from cells of a solid tissue—either in-vivo or ex-vivo—and using same for determining a cellular-components' profile of said tissue as means for identifying or characterizing: (a) abnormality of, or within, said tissue or (b) a disease state of the subject, e.g., at a tissue other than that directly tested. Accordingly, the devices and methods can provide samples that can be fed into standard and clinical workflows for performing diagnosis, such as differentiating between a normal and a diseased tissue, e.g., a tumor. The method is based on the extraction of the cellular components from cells of the tested tissue using electric field assisted biopsy (EFAB) which comprises: (i) placing at least one electrode within said solid tissue, or in proximity thereto; (ii) applying a PEF via said at least one electrode to induce permeabilization of cells of said solid tissue, and consequently release of at least one cellular-component therefrom to an interstitial space in tissue or supernatant under in vitro conditions.; (iii) extracting said at least one cellular component from said extracellular matrix (such as extracting the at least one cellular component into at least one of the at least one electrode; and optionally (iv) identifying/analyzing the at least one cellular component extracted (via techniques available to an art worker in, for example, molecular biology) so as to identify/determine the presence and type of abnormality within said solid tissue or identify/determine the presence of a disease state of the subject. These steps (i-iv) can be repeated multiple times at the same or different location and/or at the same general time or different times.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### PEF

PEF is a process consisting of applying short pulses of high voltage at high frequency, leading to biological tissue permeabilization. The term “pulsed electric field (PEF)” as used herein thus refers to the application of a pulsed electric field characterized by specific voltage, electric field strength, pulse duration, number of pulses, and pulses frequency so as to achieve biological tissue/membrane permeabilization via formation of pores on the cell membrane (electroporation).

In some embodiments, the PEF is characterized by:
(i) pulse number of from 1 to about 10,000, e.g., from 1 to about 500, from 500 to about 1000, from about 1000 to about 2000, from about 2000 to about 3000, from about 2000 to about 4000, from about 4000 to about 5000, from about 5000 to about 6000, from about 6000 to about 7000, from about 7000 to about 8000, from about 8000 to about 9000, or from about 9000 to about 10000;
(ii) pulse duration of from about 50 ns to about 10 ms, e.g., from about 50 ns to about 500 ns, from about 500 ns to about 1 ms, from about 1 ms to about 2 ms, from about 2 ms to about 3 ms, from about 3 ms to about 4 ms, from about 4 ms to about 5 ms, from about 5 ms to about 6 ms, from about 6 ms to about 7 ms, from about 7 ms to about 8 ms, from about 8 ms to about 9 ms, or from about 9 ms to about 10 ms;
(iii) electric field strength, which need not be symmetric across the positive and negative waveform. One phase of the pulse may have a higher peak when compared to the immediately succeeding pulse. This would be dependent on whether the intent is to polarize, electroporate or stimulate by depolarization. In general, the negative phase of the pulse would have a longer duration than the positive phase. This will drive electrophoresis of negatively charged material (e.g., RNA, DNA and most proteins) from the cell to assist collection of desired material. Examples of electric field strength are from about 0.1 to about 100 kV/cm, e.g., about 0.1 to about 0.5 kV/cm, about 0.5 to about 1 kV/cm, about 1 to about 5 kV/cm, about 5 to about 10 kV/cm, about 10 to about 20 kV/cm, about 20 to about 30 kV/cm, about 30 to about 40 kV/cm, about 40 to about 50 kV/cm, about 50 to about 60 kV/cm, about 60 to about 70 kV/cm, about 70 to about 80 kV/cm, about 80 to about 90 kV/cm, or about 90 to about 100 kV/cm;
(iv) biphasic, high frequency pulses of from about 1000 Hz or higher, including of from about 1kHz to 500 kHz; and
(v) pulse phase, including biphasic pulses where t1, t2, t3 and t4 are variable. The variable timing of the pulse may be synchronized to the suction duration to further promote combinatorial to augment extraction of material. The durations will be exploited to maximize release from cells before using the second phase of the pulse to induce extraction.

As would be clear to any person skilled in the art, the particular characteristics (properties) of the PEF treatment applied, i.e., the combination of particular pulse number, pulse duration, electric field strength and pulse frequency selected, may affect the efficiency of the process, e.g., the electroporation efficiency, and consequently the amount and/or types of cellular components released from the electroporated cells. The particular characteristics of the PEF treatment applied should thus be selected such that the permeabilization induced and consequently the release of the cellular component(s) would provide a cellular components profile best reflecting the cells of the target solid tissue.

### PEF Electrodes

The electrodes are generally designed to penetrate tissue (needle like) and are hollow tubes (constructed of metal or plastics) with one or more side openings (perforated needle), through which cellular material can pass. The hollow tube can be coated/covered with one or more coatings, such as an adjustable sleeve (one that can be moved up or down so as to reveal one or more additional openings on it sides). The coating can be insulating sleeve that can be adjusted to alter the side hole profile. The sleeve adjustment will control volume and rate of suction/sample collection.

In one embodiment, the at least one electroporation-electrode is hollow, and the at least one cellular component released to the extracellular matrix is extracted in step (iii) by suction via said at least one hollow electroporation-electrode (through an opening at the bottom or through the one or more side holes in the hollow tube) by a suction unit.

The electrode is designed to be associated with an electric generator to generate the PEF within the sold tissue, as available to an art worker. In some embodiments, the suction unit further comprises a collection vessel (such as a syringe or tube) for holding the extracted cellular elements.

### Additional Embodiments of the Device

Device will have a needle with a diamond tip, the needle will be 19-25 G in size.
   Needle tip may feature perforations through the shaft. The holes may be helically or radially arranged. A hole at the end will be present.
   Hole size can be from 10-500 microns in size.
   In some embodiments, the needle can be made of a porous material.
   The needle can also consist of multiple, separate cylindrical entities instead of being a single solid shaft.
The device will have a body that will connect the distal (needle) and proximal (physician) ends of the device
   The body will be of an insulating material such as polymers, PET, Pebax, nylon etc.
   The body will be flexible and have a diameter ranging from 3-6 Fr, or 1-2 mm in size.
   The length of the body will variable based on application, ranging from 5-15 cm for percutaneous procedures to 60-150 cm length for endoscopy procedures.
   The body may feature a sliding sleeve that can be used to expose portion or whole of the needle to control the region delivering electrical energy.
The distal end of the device will feature a hub
   The hub will provide a connector for introducing the electrical connector for delivering PEF to the distal tip.
   The hub will provide a connector for aspiration materials through the body and tip.
   The connector will have a three-way system for lavage of samples by infusion of a material that enhances preservation of RNA
   The connector will be part of the handle that allows manipulation and position of the tip of the device by handles (steering and positioning in 2 axis).
   The connector may feature a pressure sensor for monitoring and controlling the retrieval of samples.
   The connector will provide access to the body of the device for navigation using guidewires etc.
The device will have an electrical conduit in the form of solid, insulated shaft with an electrically active tip that will deliver energy to the distal needle of the catheter device
   The conduit will provide connection to the generator and will be used only electric pulses have to be deliver to the tip, and removed from the catheter while accessing samples.
   The conduit may feature a single or multiple solid connectors at the tip to allow delivery of pulses from a single location in the needle or the entire needle.
The device may also have additional components such as a guidewire or rigid stylet to enable introduction into the anatomy without buckling.
Include
   The device may feature infusion of a material to improve PEF delivery to the region by e.g., modulating the local osmotic properties
   The device may feature infusion of a second material (e.g., Polyvinyl Sulfonic Acid) that promotes the preservation and extraction of material in sample.

### Cancer

Solid tissues and tumors can be biopsied/sampled. These tumors can be benign or malignant (cancerous). Cancerous tumors include tumors from cancers that include, but are not limited to, sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, gastrointestinal (GI) cancer, genitourinary (GU) cancer, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

Cancer can also be monitored over the course of cancer treatment, such before, during and after chemotherapy or radiation treatment, via biopsy methods and devices disclosed herein.

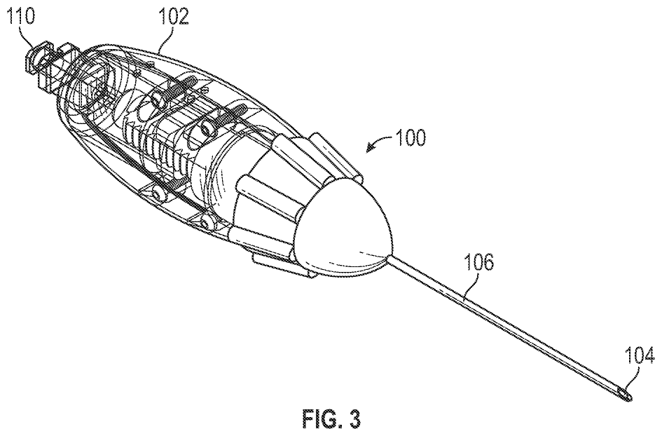

An example of a device to use for EFAB is shown in FIG. 3. As shown in FIG. 3, device 100 includes handle 102. Needle structure extends from handle 102. Needle structure 102 includes inner needle 104 and outer needle 106. Outer needle circumscribes inner needle 104. Inner needle 104 is also an electrode that can apply a current to the tissue. An insulator is deployed between inner needle 104 and outer needle 106. A tissue sample can be aspirated through inner needle 104. Relative to outer needle 106, inner needle 104 can extend and retract. The extension and/or retraction of inner needle 104 can be controlled by knob 108 or a similar device by a user.

### EXAMPLES

The following example are provided in order to demonstrate and further illustrate certain embodiments and aspects of the present invention and are not to be construed as limiting the scope thereof.

### Example I

### Introduction

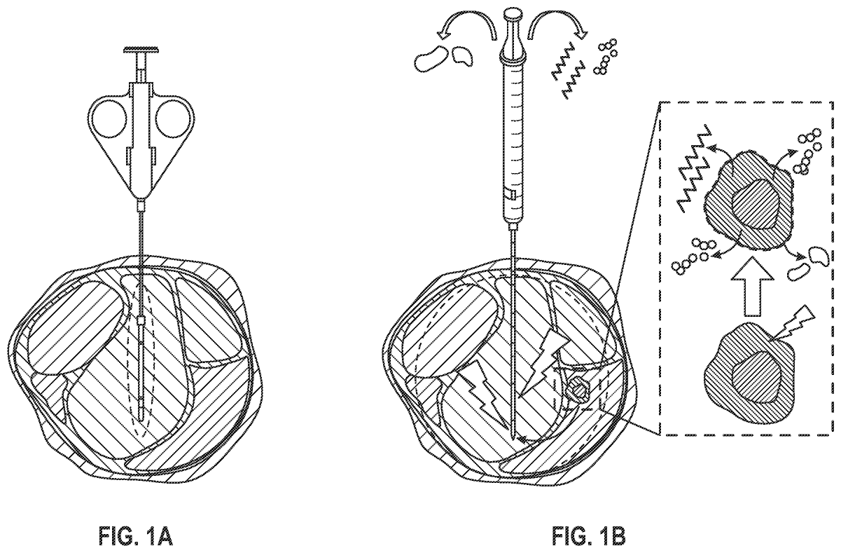

Critical cancer care decisions are based upon a view through a keyhole: patients suspected to have cancer undergo a medical procedure called biopsy where long, thin needles are used to collect a small tissue sample of the suspicious mass which is then interrogated to determine malignancy. The clinical practice of biopsy typically samples less than 1% of the tumor volume (FIG. 1A) whereas tumors are known to exhibit remarkable spatial heterogeneity, with intra-tumoral variation in protein markers, gene expression, and the presence and status of immune cells.

Recent evidence suggests that intra-tumoral heterogeneity is a key determinant of treatment response, and a prognostic factor for metastatic disease and patient survival. Surgical resection is currently the only approach that allows complete profiling and characterization of the entire tumor volume, which is infeasible for most patients, especially so at the point of initial diagnosis.

Despite major advances in cancer diagnostics and therapy, the practice of biopsy has remained unchanged over several decades, fundamentally limiting our ability to study, understand, and treat cancer.

### Results/Discussion

Provided herein is a method to advance the survival and well-being of cancer patients by with technologies that improve the precision and accuracy of disease diagnosis. Provided herein needle-based volumetric tumor profiling by providing mathematical models and technology for electric field assisted biopsy (EFAB).

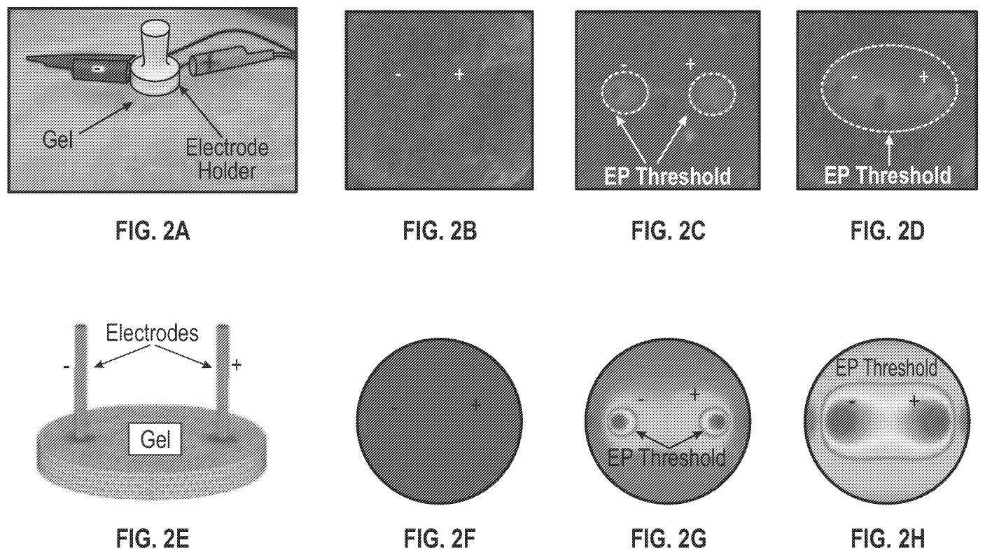

Electroporation (EP) is a technique used in the lab and clinic primarily for intracellular gene and drug delivery by application of ultrashort electric pulses using needle electrodes that transiently (˜10 minutes) alters the barrier function of the cell membrane. Cells undergoing EP can leak intracellular proteins and nucleic acids while the membrane is in a permeabilized state [5]. EP of an entire tumor will cause the release of intracellular material into the interstitial space which can be extracted by gentle suction and then used to construct a volumetric profile of the tumor (FIG. 1B). FIG. 2 demonstrates (i) dose-dependent sampling of ribonucleic acid (RNA) and proteins by EFAB of 3D tumor models (˜15 mm diameter) in vitro and (ii) that finite element models (FEM) can determine the volume sampled (FIG. 2).

### Model to Predict Volumetric Biopsy and Sampling Conditions During EFAB

EP and negative pressure demonstrate gradient effects, where respective efficacy in release of intracellular contents and transport of the material decreases as a function of distance from the electrode/suction needle interface, resulting in non-uniform representation of the tumor volume within a sample. Provided herein is a computational model to both map and optimize the EFAB biopsy sample to the tumor volume being profiled. Factors that can be used to model include mapping the electric field gradient using numerical techniques that solve for the Maxwell's equation.

It was also found that EFAB induces an EF in tissue, along with hyperthermia and cell death dependent on duration of pulse application, numerical models can inform selection of parameters to retain desirable effects while minimizing others. The equations below can be used:

$$ψ ( t ) = {\int }_{t = 0}^{t = τ} ξ \cdot exp ( \frac{- {E}_{a}}{R \cdot T ( t )} ) dt$$

$$S = \frac{N}{{N}_{0}}$$

$$S = \frac{1}{1 + exp ( \frac{E - {E}_{c} ( n )}{A ( n )} )}$$

Additionally, it was found that smaller cells (such as those in blood, red), exhibit different behavior compared to larger ones (tumor, green) that can be exploited to selectively sample cells. Equations used to model this include the following.

$$TMV = \frac{3}{2} ER cos ( θ ) ( 1 - e \frac{- t}{τ} )$$

$$τ = R {C}_{m} ( {ρ}_{i} + \frac{{ρ}_{e}}{2} )$$

Additional equations can include:

$$\vec{Q} \text{?} = \vec{∇} \cdot ( {σ}_{e} \vec{E} + \vec{{J}_{e}} ) \qquad [ 1 ]$$

$${S}_{e} = \frac{1}{1 + {e}^{E - {E}_{c} ( n )} / A ( n )} \qquad [ 2 ]$$

$${σ}_{e} = {σ}_{0} \cdot ( 1 + A \cdot H ( \vert \vec{E} \vert - {E}_{\partial } , {E}_{s} ) \qquad [ 3 ]$$

$$\vec{Q} h = ρ {C}_{p} \frac{\partial T}{\partial t} + ρ {C}_{p} \vec{u} \cdot ∇ T + \vec{∇} \cdot ( k ∇ T ) \qquad [ 4 ]$$

$$Ω ( t ) = {{\int }_{0}}^{t = τ} ζ \cdot {e}^{- {E}_{a} / R \cdot T ( t )} dt \qquad [ 5 ]$$

$${S}_{t} = 100 \cdot ( 1 - {e}^{- Ω ( t )} )$$

$${\vec{Q}}_{m} = \frac{\partial }{\partial t} ( {ε}_{p} ρ ) + \vec{∇} \cdot ( ρ \vec{u} ) \qquad [ 6 ]$$

$$\frac{\partial {c}_{i}}{\partial t} = {D}_{i} {\vec{∇}}^{2} {c}_{i} \qquad [ 7 ]$$

$$\text{?} \text{indicates text missing or illegible when filed}$$

### Computational Model

FEM models have been updated to incorporate (i) calculations for Darcy's Law and Starling's Law for flow through porous interfaces (interstitial space) and (ii) stochastic computation of EP efficacy as a function of a function of electric field gradient and treatment duration. The electrical and mechanical properties of the tumor were be parametrized to reproduce heterogeneities observed in vivo due to local variations in stromal and cellular content. Variation of suction (timing relative to EP, duration, duty cycle and peak pressure) parameters were tested on a library of in silico tumor models to determine the optimal set that maximizes yield for a given tumor volume.

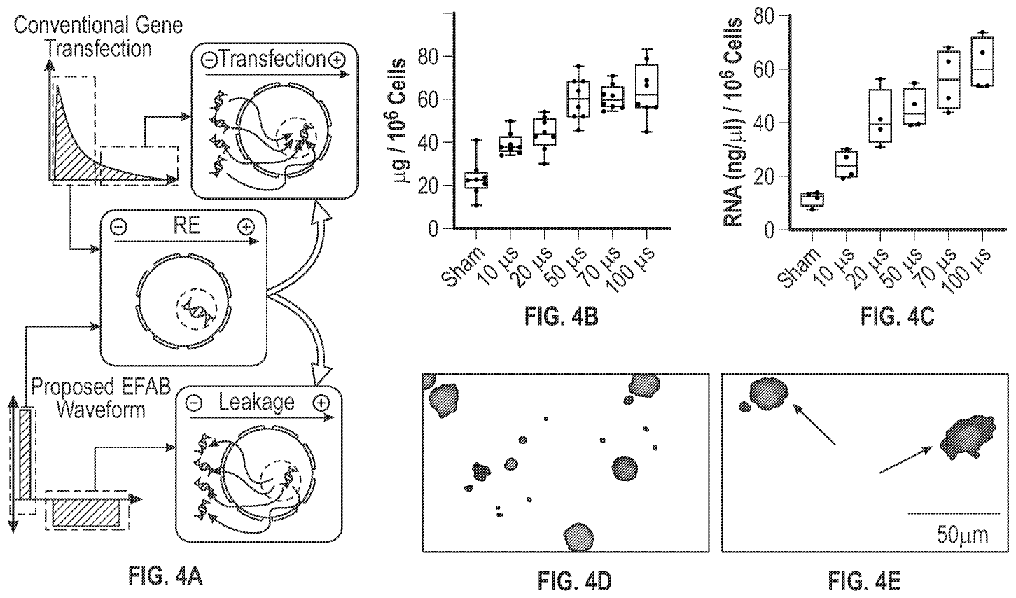

Specific waveforms for EFAB: The pulse waveform used for RE during gene and drug delivery is designed for membrane permeabilization and electrophoretic propulsion of negatively charged DNA into the cells (FIG. 4a). These waveforms were optimized by balancing the applied voltage (V) and the pulse duration (pw) that maximizes RE, while minimizing the total energy (P) injected into the tissue and the associated heating. Studies have shown that membrane permeabilization by itself to be insufficient for DNA transfer into cells, where electrophoresis substantially increased efficacy. This is accomplished during conventional RE by sequentially applying short high-strength pulses for membrane permeabilization, followed by a long low-strength waveform for electrophoretic DNA transfer. As EFAB follows the inverse process, we have identified specific waveforms that can improve transfer of materials out of the cells. We have tested the relationship between intracellular material release during EFAB to the pulse width, while using fixed values for other pulse parameters. It was found that the release of intracellular RNA, DNA and proteins was positively correlated to the pulse width (FIG. 4b-c), without any tangible impact on cell viability or membrane permeabilization efficiency. Subsequently, we tested the effect of sequentially stacking short- and long-pulse width waveforms on material release from cells loaded with an intracellular dye (FIG. 4d-e). It was found that our proposed mixed waveforms produced greater dye release into the extracellular space when compared to conventional RE waveforms.

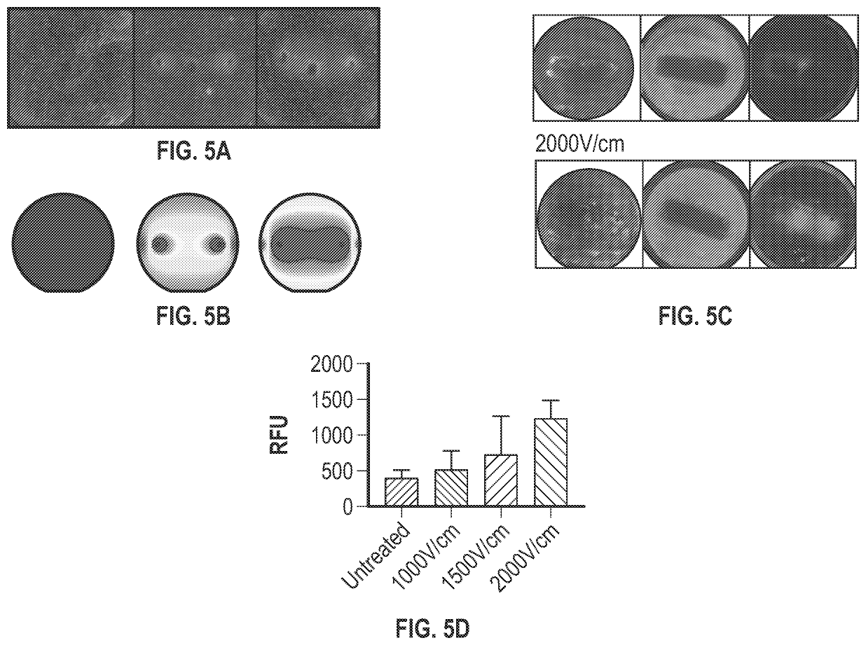

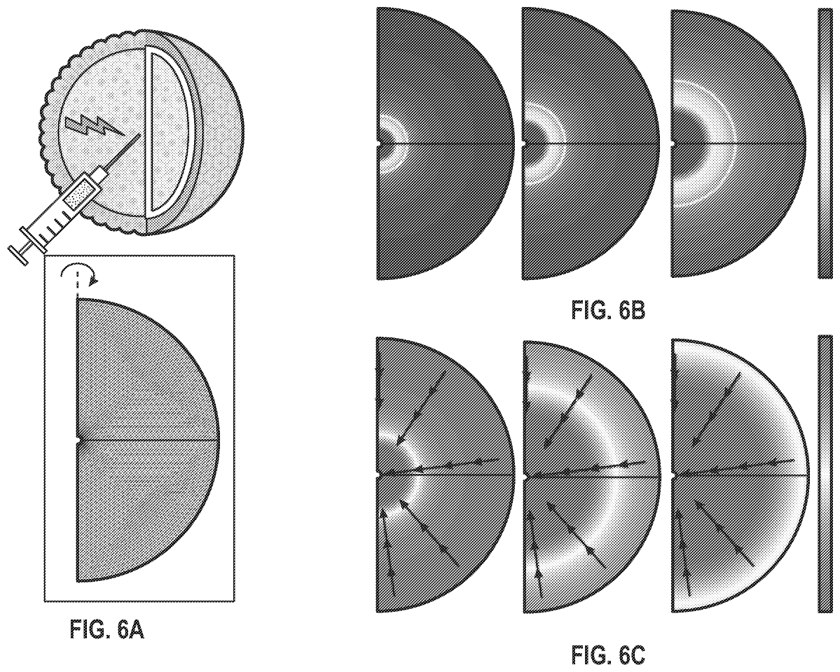

Simulation models combining electric field and suction gradients to identify optimal EFAB parameters: The volume of tissue within which intracellular material is released during EFAB is directly correlated to the electric field gradient for any given waveform. The demarcation between the region of material release and unperturbed tissue is sharp, falling within a sub-millimeter range. The use of Maxwell's equations to estimate EF in a tissue, and predicting the region of RE or IRE is well established, which we have adapted for EFAB. The intracellular material released into the interstitial space is subject to biophysical processes of diffusion and transport under suction. Application of a negative pressure to acquire this sample induces a second overlapping gradient in the tissue that ultimately determines its composition, and the tissue regions that are represented. Therefore a simulation model coupling electromagnetic and transport equations is necessary to select electric field and suction parameters to extract material from a target tissue volume by EFAB. To accomplish this electric field gradients were used to map the region of an in vitro tumor mimic undergoing permeabilization, thereby identifying the isoline delineating the region of intracellular material release (FIG. 53a-b). This model was validated by titrating the EF in a two-part tumor mimic gel, demonstrating that at fixed suction parameters, the quantity and composition of EFAB sample correlated to the electric field gradient (FIG. 5c-d). Corresponding computational models of Darcy's equation were established to estimate interstitial fluid flow characteristics during suction (FIG. 6. These combined models allows us to screen and select optimal PEF and suction parameters for EFAB from pre-defined tissue or tumor volumes.

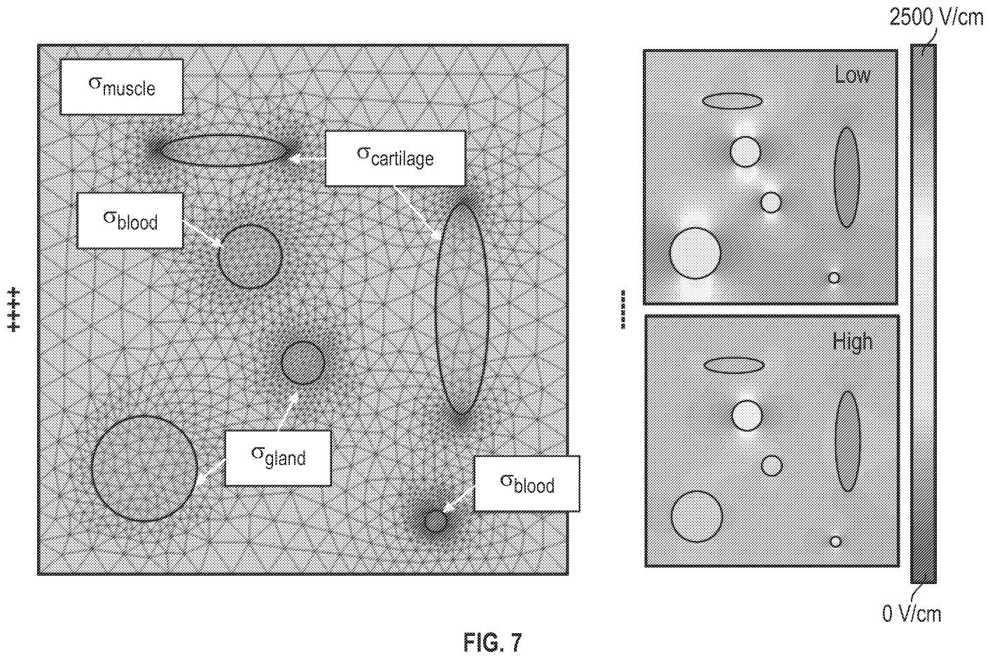

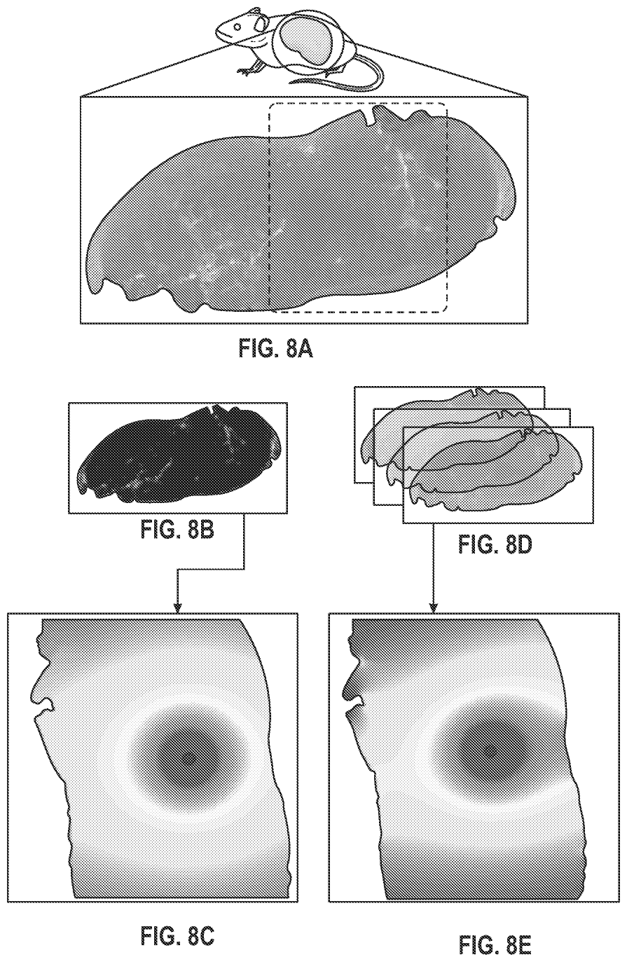

PEF Frequency selection for EFAB: Intratumoral heterogeneity within the TME is observed in physical properties such as electric conductivity(s), porosity and diffusion characteristics. Such heterogenous properties arises from both physiological gradients the presence of anatomic structures such as blood vessels and stroma. Most simulation models of existing PEF applications utilize a “bulk approach”, and do not incorporate such variations in intratumoral physical properties. To optimize EFAB parameters, we have used FEM simulations to define the effect of microscopic variations in s within the TME on electric field strength, and its impact ablation treatment outcomes. The s of biological tissues is sensitive to the frequency of the electrical waveform, where increasing the frequency can reduce the variations in electric conductivity within the TME. Simultaneously, using a high frequency waveform also reduces off-target neuromuscular stimulation during PEF application, with additional benefits when using a biphasic waveform such as what we have defined for EFAB. We then tested the impact of increasing PEF frequency in a simulation model containing geometric features and heterogenous tissue having varied electrical conductivity, both of which are known to impact the electric field strength distribution. As anticipated, we found substantial reduction in electric field strength heterogeneities within the TME, proportional to increasing PEF frequency (FIG. 7 with best results in the 50-500 kHz range. We have also established a workflow for extracting key features from the TME (stroma, blood vessels, necrotic and viable regions) from H&E-stained slides by color deconvolution and segmentation to extract regions with vasculature, stroma, viable cells, and necrosis. Appropriate electrical conductivity was assigned to these regions to construct a mesoscale electric field map for use with EFAB simulations (FIG. 8).

Electric field gradient and related effects modeling: The model for EFAB incorporates the physics equations provided herein above (1-7) to describe membrane permeabilization by electric field gradient (E) and by stochastic estimates using a time-dependent (i) Laplace representation of Maxwell's equations for electricity (magnetic components assumed to be zero) and by a (ii) modified Peleg-Fermi equation to estimate the proportion (Sₑ) of cells that will undergo permeabilization, cell death or stimulation. Membrane permeabilization induced dynamic, spatial alterations in tissue conductivity ( ) was implemented using a (iii) Heaviside function. Temperature changes by (iv) Joule heating (T) from passage of current and corresponding incidence of (v) thermal injury (Sₜ) are represented by metabolic heat and Arrhenius equations respectively. These equations are minimum necessary to estimate select appropriate parameters for stimulating material release from a defined tissue or tumor volume.

Coupling with mass transport model: The negative pressure gradient (Dp) from suction, and its effect on interstitial fluid flow and material diffusion are modeled by coupling (vi) Brinkman-Darcy Equation and (vii) Fick's law. The permeability of the medium may be coupled to the suction gradient by an inverse exponential function to improve model accuracy. The results from the electric field model can be used to establish the initial conditions for concentrations gradients based on estimated region of permeabilization and temperatures. The coupled suction model can then be used to derive optimal parameters to maximize material extraction specifically from the region stimulated by the electric field.

### Example II

### Introduction

Checkpoint inhibitor immunotherapy (CII) targeting PD-1/PD-L1 signaling has been shown to substantially prolong the survival of patients with metastatic renal cell cancer (mRCC)(1). Yet, fewer than 25-42% of mRCC patients receiving CII respond to treatment despite being selected on basis of positive PD-L1 expression. mRCC exhibits significant intra-tumoral genomic and cellular heterogeneity, which can influence intra-tumoral levels of PD-L1, a dynamic protein biomarker that whose expression is induced and influenced by multiple factors in the tumor microenvironment. Determining PD-L1 expression profile of a whole tumor concomitant to immune cell activity can improve its utility as a biomarker and enhance the individualized care of mRCC patients by identification of those most likely to benefit from CII. Surgical removal of tumors for such profiling is not clinically feasible, while the primary source of tumor tissue for assessing PD-L1 expression is from percutaneous tumor biopsy, which is fundamentally limited by the small volume of tumor that can be sampled for analysis.

This work will demonstrate that proteomic and transcriptomic analysis of intracellular proteins and RNA extracted from the entire tumor volume using electroporation will enable whole-tumor PD-L1 profiling. Electroporation (EP) is a minimally invasive clinical technique where ultrashort electric pulses are applied using thin needle electrodes to introduce genetic material into cells by transiently permeabilizing the cell membrane. Cells can leak intracellular material following EP, which has been exploited to extract RNA from single cancer cells or using microfluidic approaches (5), the feasibility of extending this approach for interrogating bulk tissue or a whole tumor is novel. During unrelated experiments, it was discovered that the presence of intracellular proteins in media following EP of large (˜14 mm) 3D tumor mimic in vitro. Bulk transcriptomic and proteomic analysis of intracellular contents released from kidney tumors by EP will reveal (i) whether PD-L1 expression is the result of immune recognition (INF-γ induced); (ii) intra-tumoral spatial distribution and levels of PD-L1 expression (mRNA vs. protein); and (iii) the relative contribution of non-INF-γ pathways.

### Materials and Methods

3D tumor mimics will be constructed in 24 well plates by embedding murine (RenCa) or human (786-0, A498, selected based on endogenous high or low PD-L1 expression) cancer cells in a collagen gel matrix, and perform EP (8 pulses, 100 μs pulse width) covering 25-100% of the tumor volume by increasing the applied voltage (500-2000 V), determined via computer models. Immune recognition will be simulated through exposure to recombinant INF-γ at increasing dose. Conditioned media from EP±INF-γ treated tumors and controls will be evaluated for total RNA (extracted by aspiration and stabilization with TRIzol LS) and protein content (BCA assay). RNA will be treated with DNasel prior to RT-qPCR assays and quality established with Qubit and Agilent Pico assays. Change in PD-L1 mRNA (using RT-qPCR with TaqMan probe-based assays), and the levels of STAT1 and IRF1 (ELISA, related to INF-γ exposure) will establish response to simulated immune activity. Quantity of PD-L1 mRNA in media will be compared to the volume of EP treated tumor, and the total PD-L1 protein in tumor (western blotting) as well as its spatial distribution (immunofluorescence). Relative contribution of immune-activity and constituent pathways to PD-L1 expression will be determined by quantifying the relative phosphorylation of STAT1 with MEK/ERK, PI3K/mTOR in media from EP+INF-γ treated tumor and control. Results will be validated in vivo by implanting Balb/cJ mice (n=10/cohort) with subcutaneous RenCa tumors, performing EP with caliper electrodes and fine needle aspiration of intra-tumoral contents for analysis.

### Device

The development of a novel tool (electroporation) for the extraction and harvest of protein and RNA from whole or partial tumor volume for proteomic and transcriptional interrogation to reveal factors underlying intra-tumoral PD-L1 expression, as an example, is a novel, non-obvious device and method, leading to a novel and powerful diagnostic procedure to support checkpoint inhibitor immunotherapy (CII) and targeted therapy of kidney cancer patients.

### New Paradigm in the Diagnostic Continuum for Kidney Cancer and Other Malignancies

Kidney cancer can demonstrate substantial intra- and inter-tumoral genomic heterogeneity, with implications for immune cell activity and subsequent expression of induced proteins such as PD-L1 (4). This also underscores the obvious limitations of standard of care core needle biopsy or fine needle aspiration as the primary source of material for tumor profiling and diagnostic decision making (FIG. 1A). Recently developed techniques such as single cell sequencing and blood-based liquid biopsies for CTCs, ctDNA and mRNA have greatly improved the ability to study and understand kidney cancer and other malignancies. Yet, there exist gaps in the diagnostic continuum as core needle biopsies are often the source material for single cell sequencing analysis, and liquid biopsies provide a snapshot of systemic disease which can be influenced by tumor or cancer specific biologic characteristics (some tumors or tumors sites can contribute more material to the sample from the blood pool). The method provide herein (FIG. 1B) advances the ability to profile whole or partial tumor volume, providing a novel tool and method that can further narrow gaps in the ability to characterize, understand and study kidney cancer and other solid tumors.

### PD-L1 as a Biomarker

PD-L1 expression in cancer cells and tumor associated macrophages was considered to occur in response to INF-γ secreted by immune cells, and the inhibitory effect of PD-L1 on T-cell activity provided rationale for its application as a biomarker for CII. However, subsequent investigations have revealed PD-L1 expression can be constitutive and may also arise in response to factors such as hypoxia which are unrelated to immune cell activity. Paradoxically, PD-L1 inhibition has been shown to yield therapeutic effect without immune cell activity in some cancers, and at the same time patients with a low PD-L1 score have demonstrated T cell mediated clearance of their tumors. These findings signify the importance of the context in which PD-L1 expression occurs, which should be taken into consideration when using it as a therapeutic biomarker. This cannot be easily captured by the current clinical practice of IHC based PD-L1 analysis, reinforcing the need for an integrated proteomic and transcriptional approach.

### Clinical Applications for Electroporation (EP)

EP is used in patients for the focal ablation of small tumors, including kidney cancer, and for the delivery of drugs or genetic material. EP is a minimally invasive technique performed using thin needle electrodes, similar in size and shape to percutaneous devices used for core biopsy. Provided herein is a novel clinical application of EP technology by shifting focus from cancer therapy to its use as a diagnostic tool. While cells permeabilized with EP are well known to leak intracellular content like proteins or RNA, the application of this technique has been limited to single cells or microfluidic devices. It was shown that EP can release intracellular material from large 3D tumor mimics in vitro, sufficient for interrogation with molecular biology techniques. Extraction efficiency can be further increased whilst preserving cell viability by use of physiochemical techniques that enhance the osmotic gradient and reduce RNA degradation.

### Example III

Preliminary testing indicated that a multi-hole Profusion needle to be good candidate for our device. It was rationalized that using an off the shelf needle as part of our kit was expedient as it saved considerable time and resource involved in manufacture of such a device.

Following multiple experiments, it was decided to proceed with a two needle system for the device. The outer needle served as the conduit to access the tumor while also controlling the length of electrode within the tumor. The inner needle, based on a Chiba needle, served as both the electrode and the means for material extraction. This was enabled by having a removeable electrical connector to the device, thus maintaining the lumen open when not passing electricity.

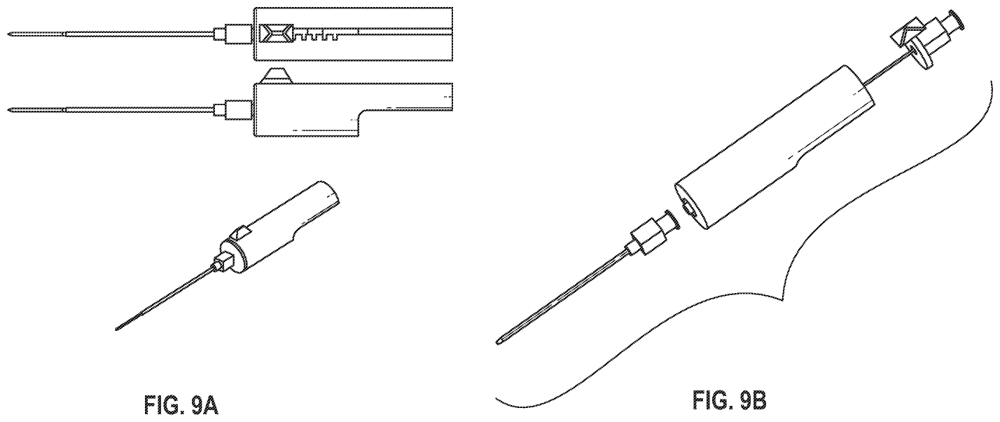

A custom positioning system was designed to couple the two needles while allowing easy access to the inner needle for manipulation/sample extraction (FIG. 9).

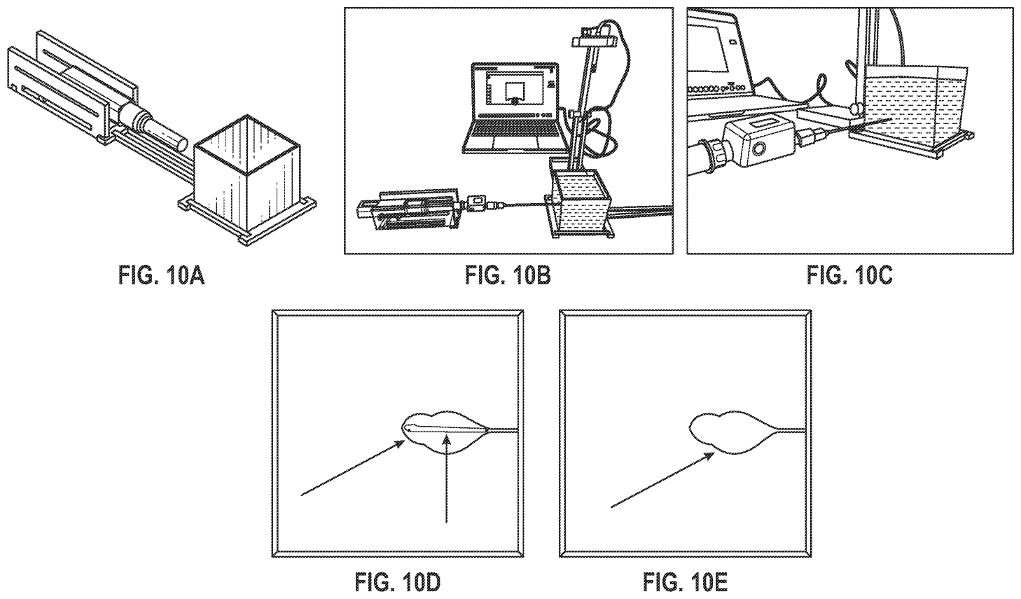

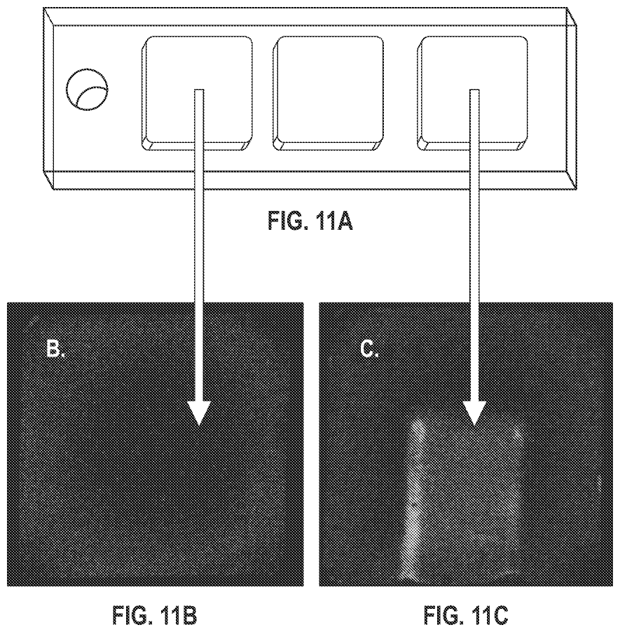

Preliminary testing of the chiba needle for infusion studies showed the ability for reproducible/predictable material deposition while maintaining constant pressure. It is anticipated that these results will transfer for future suction applications as well (FIG. 10). Likewise, electrical testing showed that the needles to capable of delivering the desired energy into 3D tumor mimic constructs, with clean delineation of the region of effect (FIG. 11).

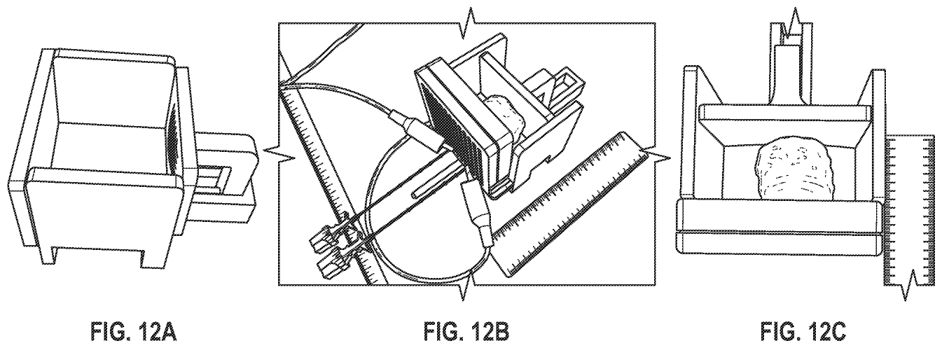

A 3D printed/laser cut fixture was established that allowed positioning of a patient prostate in an anatomically correct fashion while also mimicking the workflow of standard of care transperineal biopsy. This fixture was used to validate the device set up in 3 patient prostates. The material collected from these prostates was submitted for quality control and sequencing at the genomic core facility. The RNA and DNA samples from the first prostate passed quality control for sample quantity and quality for sequencing. The other samples are pending evaluation (FIG. 12).

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

### Summary of Aspects

In some aspects, the techniques described herein relate to a device including: (i) at least one electrode associated with an electric generator to generate a pulsed electric field (PEF) in solid tissue, wherein said electrode includes more than one hole/opening at the end in contact with said solid tissue and an insulating sleeve that can be adjusted to alter the side hole profile; and (ii) a cellular-component extraction element by suction through at least one electrode, wherein upon introducing said at least one electrode into said solid tissue, and generating the PEF, the PEF induces a biophysical response from cells in solid tissue or other condition (such as be blood, in vitro etc.) resulting in at least one cellular component to exit to extracellular matrix which is then extracted by said extraction element.

In some aspects, the techniques described herein relate to a device, wherein the at least one electrode includes a needle.

In some aspects, the techniques described herein relate to a device, wherein the needle is an inner needle and the device further includes an outer needle at least partially encompassing the inner needle.

In some aspects, the techniques described herein relate to a device, wherein the inner needle is configured to extended outwardly relative to the inner needle.

In some aspects, the techniques described herein relate to a device, wherein the needle is adapted for aspiration.

In some aspects, the techniques described herein relate to a device, wherein the inner needle is configured to be extend outwardly under the control of a knob.

In some aspects, the techniques described herein relate to a device, wherein the needle can be retracted under the control of the knob.

In some aspects, the techniques described herein relate to a device, wherein the device includes an insulator disposed between the inner needle and the outer needle.

In some aspects, the techniques described herein relate to a device, further including an electrical connection connected to the inner needle.

In some aspects, the techniques described herein relate to a method to extract biological material from a tissue of a subject, said method including: i) placing at least one electrode within said tissue, wherein said electrode includes more than one hole/opening at the end in contact with said tissue and an insulating sleeve that can be adjusted to alter the side hole profile; ii) applying pulsed electric field (PEF) via said at least one electrode of (i) to thereby induce permeabilization and/or other biophysical responses (such as cell stimulation) of cells of said tissue and release of at least one cellular component therefrom to an interstitial space; and iii) extracting said at least one cellular component from said interstitial space and optionally providing the extracted at least one cellular component to standard clinical diagnostic or biomedical research workflows, wherein one or more computational models are used to guide parameter selection to guide treatment and extraction parameters.

In some aspects, the techniques described herein relate to a method, wherein the at least one electrode includes a needle of a device.

In some aspects, the techniques described herein relate to a method, wherein the needle is an inner needle and the device further includes an outer needle at least partially encompassing the inner needle.

In some aspects, the techniques described herein relate to a method, wherein the inner needle is configured to extended outwardly relative to the inner needle.

In some aspects, the techniques described herein relate to a method, wherein the needle is adapted for aspiration.

In some aspects, the techniques described herein relate to a method to determine if a tissue in a subject includes a tumor, said method including: i) placing at least one electrode within said tissue, wherein said electrode includes more than one hole/opening at the end in contact with said tissue and an insulating sleeve that can be adjusted to alter the side hole profile; ii) applying pulsed electric field (PEF) via said at least one electrode of (i) to thereby induce permeabilization and/or other biophysical responses (such as cell stimulation) of cells of said tissue and release of at least one cellular component therefrom to an interstitial space; iii) extracting said at least one cellular component from said interstitial space to feed into standard clinical diagnostic or biomedical research workflows; and iv) using the standard clinical diagnostic or biomedical research workflows to identify the at least one cellular component extracted so as to determine the presence of the tumor within said tissue, wherein one or more computational models are used to guide parameter selection to guide treatment and extraction parameters.

In some aspects, the techniques described herein relate to a method, further including treating the tumor.

In some aspects, the techniques described herein relate to a method wherein the tumor includes sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, gastrointestinal (GI) cancer, genitourinary (GU) cancer, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some aspects, the techniques described herein relate to a method, wherein an accuracy of a cancer diagnosis is improved relative to a method that does not include using the electrode.

In some aspects, the techniques described herein relate to a method, further including varying a strength of the pulsed electric field.

In some aspects, the techniques described herein relate to a method, wherein the strength of the pulsed electric field is varied to release a desired target cell type.

What is claimed is:

1. A device comprising:
(i) at least one electrode associated with an electric generator to generate a pulsed electric field (PEF) in solid tissue, wherein said electrode comprises one or more hole/opening at the end in contact with said solid tissue and an insulating sleeve that can be adjusted to alter the side hole profile; and
(ii) a cellular-component extraction element by suction through at least one electrode,
wherein upon introducing said at least one electrode into said solid tissue, and generating the PEF, the PEF induces a biophysical response from cells in solid tissue or other condition (such as be blood, in vitro etc.) resulting in at least one cellular component to exit to extracellular matrix which is then extracted by said extraction element.

2. The device of claim 1, wherein the at least one electrode comprises a needle.

3. The device of claim 2, wherein the needle is an inner needle and the device further comprises an outer needle at least partially encompassing the inner needle.

4. The device of claim 3, wherein the inner needle is configured to extended outwardly relative to the inner needle.

5. The device of claim 2, wherein the needle is adapted for aspiration.

6. The device of claim 4, wherein the inner needle is configured to be extend outwardly under the control of a knob.

7. The device of claim 6, wherein the needle can be retracted under the control of the knob.

8. The device of claim 3, wherein the device comprises an insulator disposed between the inner needle and the outer needle.

9. The device of claim 3, further comprising an electrical connection connected to the inner needle.

10. A method to extract biological material from a tissue of a subject, said method comprising:
i) placing at least one electrode within said tissue, wherein said electrode comprises one or more hole/opening at the end in contact with said tissue and an insulating sleeve that can be adjusted to alter the side hole profile;
ii) applying pulsed electric field (PEF) via said at least one electrode of (i) to thereby induce permeabilization, hyperthermia, and/or other biophysical responses (such as cell stimulation) of cells of said tissue and release of at least one cellular component therefrom to an interstitial space; and
iii) extracting said at least one cellular component from said interstitial space and optionally providing the extracted at least one cellular component to standard clinical diagnostic or biomedical research workflows,
wherein one or more computational models are used to guide parameter selection to guide treatment and extraction parameters.

11. The method of claim 10, wherein the at least one electrode comprises a needle of a device.

12. The method of claim 11, wherein the needle is an inner needle and the device further comprises an outer needle at least partially encompassing the inner needle.

13. The method of claim 12, wherein the inner needle is configured to extended outwardly relative to the inner needle.

14. The method of claim 11, wherein the needle is adapted for aspiration.

15. A method to determine if a tissue in a subject comprises a tumor, said method comprising:
i) placing at least one electrode within said tissue, wherein said electrode comprises more than one hole/opening at the end in contact with said tissue and an insulating sleeve that can be adjusted to alter the side hole profile;
ii) applying pulsed electric field (PEF) via said at least one electrode of (i) to thereby induce permeabilization and/or other biophysical responses (such as cell stimulation) of cells of said tissue and release of at least one cellular component therefrom to an interstitial space;
iii) extracting said at least one cellular component from said interstitial space to feed into standard clinical diagnostic or biomedical research workflows; and
iv) using the standard clinical diagnostic or biomedical research workflows to identify the at least one cellular component extracted so as to determine the presence of the tumor within said tissue,
wherein one or more computational models are used to guide parameter selection to guide treatment and extraction parameters.

16. The method of claim 15, further comprising treating the tumor.

17. The method of claim 15 wherein the tumor comprises sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, gastrointestinal (GI) cancer, genitourinary (GU) cancer, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

18. The method of claim 15, wherein an accuracy of a cancer diagnosis is improved relative to a method that does not include using the electrode.

19. The method of claim 15, further comprising varying a strength of the pulsed electric field.

20. The method of claim 19, wherein the strength of the pulsed electric field is varied to release a desired target cell type.
